(19) United States
(12) Patent Application Publication
(10) Pub. No.: US 2017/0234307 A1
(43) Pub. Date: Aug. 17, 2017

(54) VOLUMETRIC PUMP WITH BLEED MECHANISM

(71) Applicant: SWISSINNOV PRODUCT SARL, Gland (CH)

(72) Inventors: Florent JUNOD, Veigy-Foncenex (FR); Thierry NAVARRO, Gland (CH)

(73) Assignee: SWISSINNOV PRODUCT SARL, Gland (CH)

(21) Appl. No.: 15/123,121

(22) PCT Filed: Feb. 27, 2015
(86) PCT No.: PCT/IB2015/000250

§ 371 Date: Sep. 1, 2016

(30) Foreign Application Priority Data
Mar. 2, 2014 (IB) PCT/IB2014/000225

(51) Int. Cl. F04B 7/00; F04B 1/12; A61M 5/142; F04B 1/02

(52) U.S. Cl. CPC F04B 7/0007; F04B 7/0061; F04B 1/02; F04B 1/12; A61M 5/14216; A61M 5/1422

(57) ABSTRACT

A pump having two pistons which are parallel and in opposition and are disposed in two, cylindrical, cavities in a pump body, including at least one inlet port and at least one outlet port connected to a rotational port-switching device which can move axially along its rotational axis and is held in abutment in the pump body by a restoring member, in the form of a spring.

### PRIOR ART

There are different types of reciprocating piston volumetric pumps such as those described in patents PCT/IB2009/006189 and PCT/IB2010/001683, the principle of which consists of filling one pumping chamber during the rotation of the motor and simultaneously emptying the other chamber in order to obtain a continuous flow. These systems employ an interchangeable pump that is attached to a drive composed of a motor and a mechanical assembly that controls the movement of the pistons such that it is synchronous with the movement of the port switch.

The invention relates to a volumetric pump that integrates pressure sensors and a purge mechanism, and is composed of two reciprocating pistons for dispensing liquids, medications, foods, detergents, cosmetic products, chemical compounds, or any other type of fluid, gel, or gas with precision and at a variable flow rate.

Although these pumps can be used in numerous applications, particularly in the medical field, a first problem encountered with such systems is that they do not enable the pressure to be measured in the pumping chambers directly at the pump, which is often necessary in perfusion pumps in order to detect, for example, an occlusion at the inlet or at the outlet.

A second problem is that the pumping chambers and the fluidic circuit of the pump cannot be purged without mounting the pump on the drive and starting the latter. Purging is often necessary when starting a pump in order to evacuate all of the air or any other fluid before putting a patient on an IV, for example.

### DESCRIPTION OF THE INVENTION

This invention relates to a high performance pump composed of two opposed reciprocating pistons, and integrating elements for measuring the pressure at the inlet and at the outlet as well as a purge mechanism directly on the pump.

This invention solves the problems presented in the preceding and makes a simple adjustment possible for producing, on a very large scale, disposable, inexpensive, preferably plastic pumps with an interchangeable element in contact with the fluid being pumped.

The pump comprises two parallel opposing pistons disposed inside two preferably cylindrical cavities of a pump body, with at least one inlet port and at least one outlet port having in each case at least one channel connecting to a cavity that is located on a side of the pump body and closed by a flexible element attached to the pump body. The inlet and outlet ports are also in contact with a detachable port switch held in place in the pump body by a reset element, preferably in the form of a spring.

The pressure measurement principle consists of connecting each port to a cavity closed by a flexible element fastened to the pump body and placed in contact with a second cavity located on the pump drive such that the flexible element constitutes a sealed interface between the two cavities and deforms under the effect of the pressure differential between the two cavities. Each cavity located on the drive is connected to a pressure sensor preferably disposed in the drive for measuring the pressure exerted on each flexible element and corresponding to the pressure in each pumping chamber.

The pump drive is composed primarily of a bracket, a guide carriage guided by an eccentric shaft connected to a rotor attached to an actuator, which actuator is preferably in the form of a motor.

The movement of the pistons is adapted such that it is synchronous with the switch element of the inlet and outlet ports by means of an oblong opening located on the guide carriage bearing the piston holding elements.

The pump purging principle consists of moving the port switch, preferably axially, so as to create a gap between the sealing gasket in contact with the inlet and outlet ports of the pump. The fluid can then flow freely between the inlet and outlet ports without having to connect the pump to the drive. Each piston is composed of a drive bracket, preferably with a V-shaped opening allowing the two pistons to be positioned at the top dead center, thus reducing the volume of each chamber to a minimum so that the fluidic circuit of the pump can be purged completely before the pump is connected to the drive. This function also makes it possible to hook the pump up to the drive regardless of the position of the guide carriage of the drive.

The pump is particularly well adapted for low cost production since it is made entirely of plastic parts that can easily be produced by injection molding and assembled automatically.

### DESCRIPTION OF THE DRAWNINGS

The present invention will be more clearly understood by reading the description of examples, which are given purely for illustration and which are in no way limiting, and refer to the appended drawings, wherein:

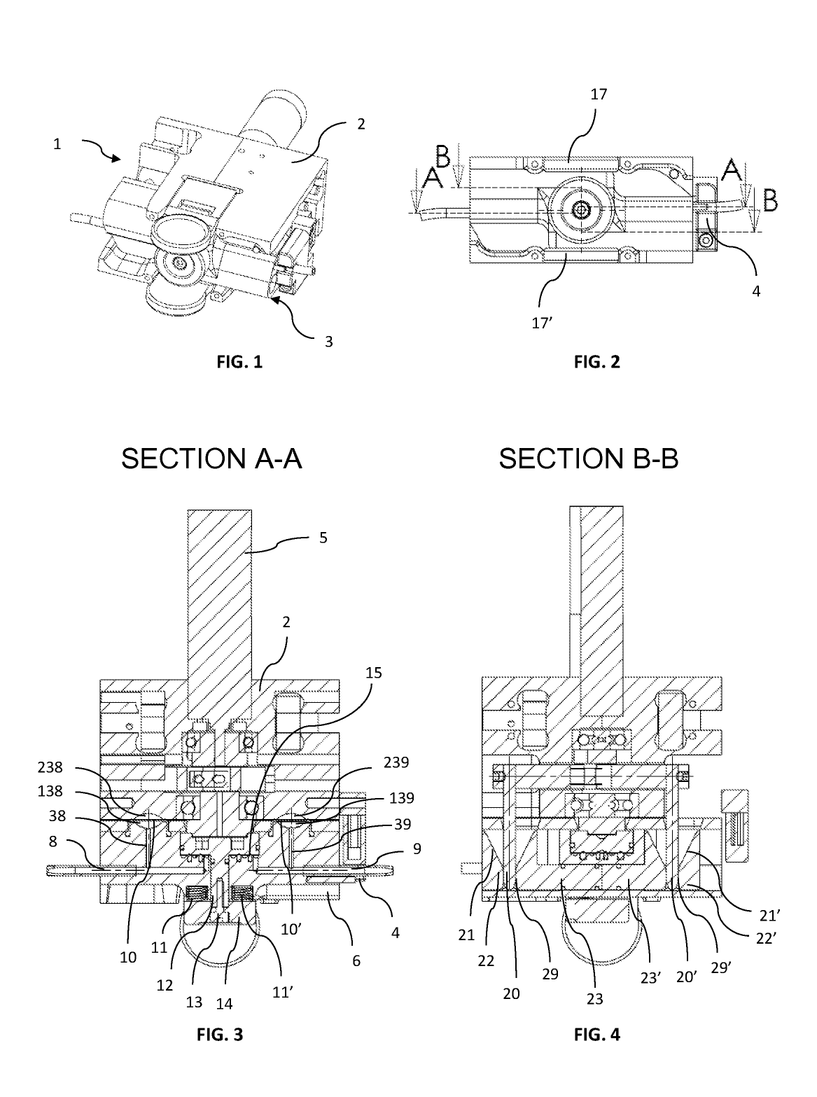

FIG. 1 is an overall view of the pump attached to a drive

FIG. 2 is a view from above of the pump attached to a drive

FIG. 3 is a longitudinal section along the line A-A of FIG. 2

FIG. 4 is a longitudinal section along the line B-B of FIG. 2

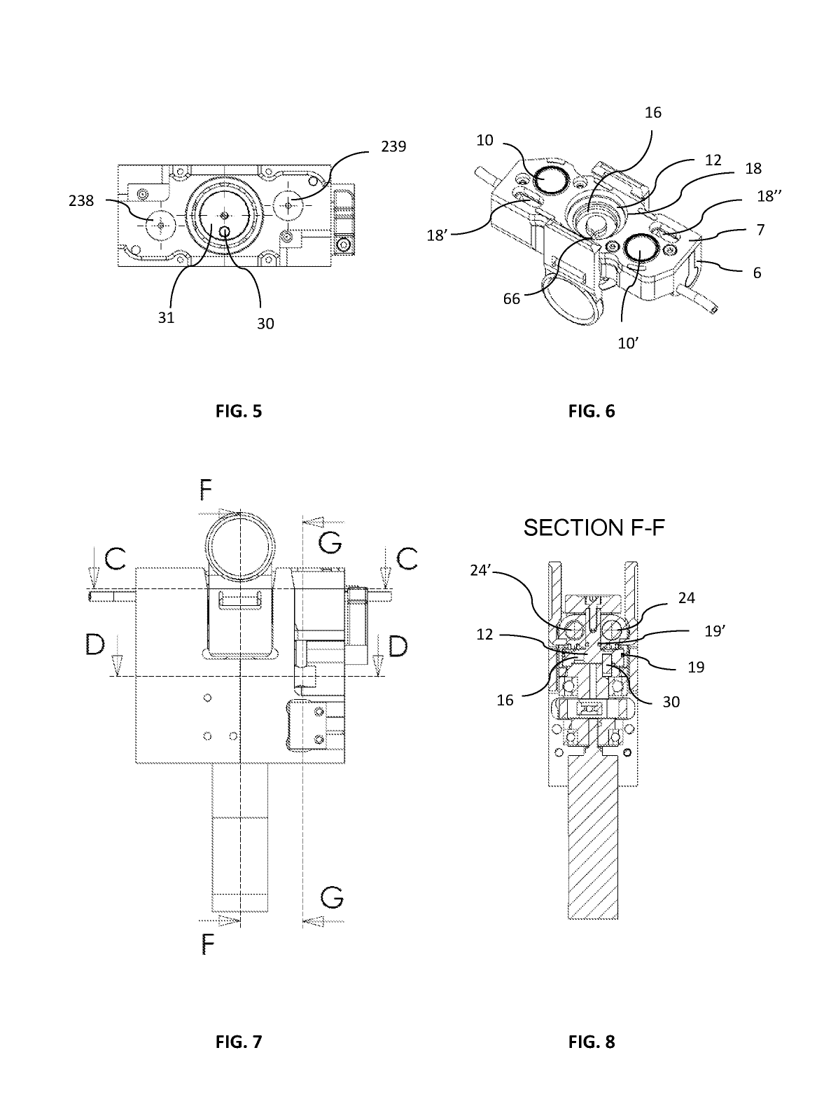

FIG. 5 is a view from above of the drive

FIG. 6 is a view from below of the pump

FIG. 7 is a side view of the pump attached to a drive

FIG. 8 is a longitudinal section along the line F-F of FIG. 7

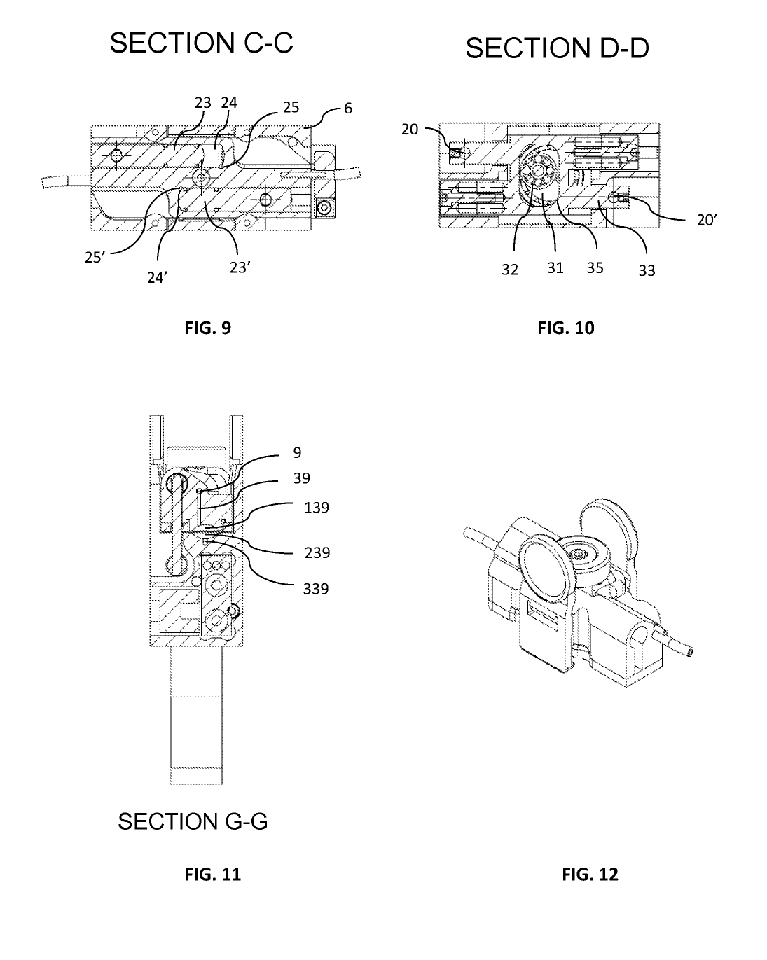

FIG. 9 is a longitudinal section along the line C-C of FIG. 7

FIG. 10 is a longitudinal section along the line D-D of FIG. 7

FIG. 11 is a longitudinal section along the line G-G of FIG. 7

FIG. 12 is a perspective view of the pump

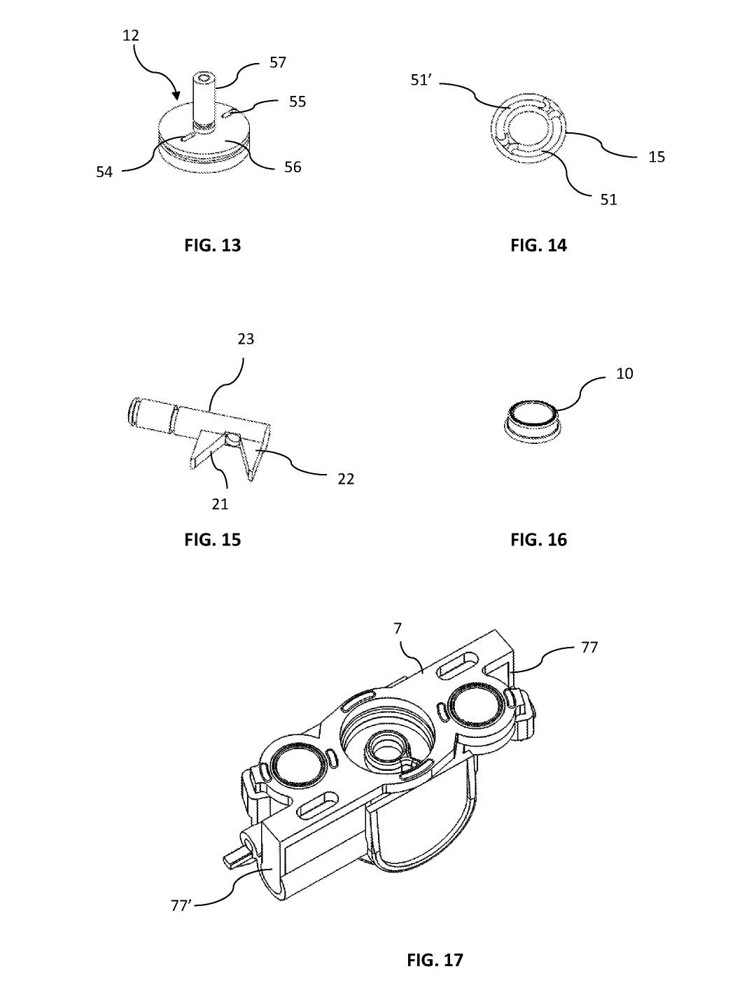

FIG. 13 is a perspective view of the port switch

FIG. 14 is a perspective view of the port switch gasket

FIG. 15 is a perspective view of a piston

FIG. 16 is a perspective view of a flexible element

FIG. 17 is a view from below of a second variant of the pump

According to FIGS. 1, 3, 6 and 9, the pump (3) comprises a cover (7) that has a preferably circular central opening (18) and is attached to the pump body (6). The cover (7) comprises two openings (18′, 18″), preferably on either side of the central opening (18). Two flexible elements (10, 10′) are fastened to the cover (7) so as to close the cavities (138, 139) in contact with the channels (38, 39) connecting the cavities (138, 139) to the inlet and outlet ports (8, 9). A port switch (12) is positioned between the cover (7) and the pump body (6) so as to compress a port switch sealing element (15) disposed between the pump body (6) and the port switch (12).

According to FIGS. 6 and 9, two opposed, parallel, preferably eccentric pistons (23, 23′) are disposed in the openings, preferably circular in cross section, located on either side of the pump body (6) so as to form two pumping chambers (24, 24′) with the pistons (23, 23′). The openings (18′, 18″) are preferably oblong and parallel to the pumping chambers (24, 24′).

According to FIGS. 3, 8, 9, 13, and 14, the inlet port (8) is in contact with the port switch (12) toward its center between the port switch gasket (15) and the preferably circumferential gasket (19′). The outlet port (9) is in contact with the port switch (12) on its periphery between the port switch gasket (15) and the preferably circumferential gasket (19). The pumping chambers (24, 24′) are in contact with the cavities (51, 51′) of the switch sealing element (15) by means of channels (25, 25′).

According to FIGS. 3, 4, 8, 13, and 14, the recesses (54, 55) located on the port switch (12) make it possible to bring the cavities (51, 51′) connected to the inlet and outlet ports (8, 9) into contact with the pumping chambers (24, 24′) in an alternating manner as the port switch (12) rotates. A guide element (57) is preferably integrated in the port switch (12).

According to FIGS. 5, 6, and 8, the port switch (12) is driven by a guide element (30), preferably in the form of a pin and situated eccentrically on the rotor (31) in order to engage in the cavity (16) positioned on the bottom side of the port switch (12). A stop element (66) located in the cavity (16) provides a support for the guide element (30) as the rotor (31) turns so as to drive the port switch (12) by rotation around its shaft. The cavity (16) is preferably as long as possible so that the pump (3) can be attached to the drive (2) regardless of the position of the rotor (31).

According to FIGS. 4, 10, and 15, each piston (23, 23′) comprises a bracket (22, 22′) with a V-shaped opening (21, 21′), ending in a hole (29, 29′) in the piston, and located perpendicular to the piston shaft. The pistons (23, 23′) are connected to the guide carriage (33) by means of holding elements (20, 20′), preferably in the form of pins, engaging in the holes (29, 29′). The holding elements (20, 20′) are fastened on either side of the guide carriage (33), which has an oblong opening (35) in its center, in which is disposed a guide element (32) preferably in the form of a roller or bearing, positioned eccentrically on the rotor (31).

According to FIGS. 3, 8, 9, and 11, each flexible element (10, 10′) forms a sealed interface between the cavities (138, 139) located on the pump body (6) and the cavities (238, 239) located on the drive (2), respectively, when the pump (3) and the drive (2) are assembled. Each cavity (238, 239) is in contact with a channel (339, and another not shown), which are each connected to a pressure sensor (not shown) to measure the pressure exerted in the cavities (238, 239) corresponding to the pressure applied to the flexible elements (10, 10′), which deform under the effect of the pressure in the cavities (138, 139), which is equivalent to the pressure in the inlet and outlet ports (8, 9) in contact with the pumping chambers (24, 24′).

According to FIG. 3, an air sensor (4) is arranged around a tube connected to the outlet port (9) so as to be able to detect the presence of air in the pump outlet. A second air sensor (not shown) can be disposed at the pump inlet in the same manner.

According to FIGS. 3 and 4, a push-piece (14) is attached to the port switch (12) by means of a fastener (13). One or two reset elements (11, 11′), preferably in the form of strips or springs, positioned between the push-piece (14) and the pump body (6) ensure(s) that the port switch (12) is held against the sealing element (15) even when the pump (3) is not hooked up to the drive (2). When the push-piece (14) is pressed by hand, the port switch (12) moves axially so as to create a gap between the sealing element (15) and the port switch (12), which has the effect of bringing the inlet and outlet ports (8, 9) into contact and thus enabling the fluid to flow freely by gravity or under the effect of pressure between the inlet and outlet of the pump. The pistons (23, 23′) can be positioned manually at the top dead center in order to reduce the volume of the pumping chambers (24, 24′) to a minimum, and it is then possible to purge the fluidic circuit of the pump (3) completely by pressing the push-piece (14) and letting the fluid flow freely between the inlet and the outlet (8, 9) of the pump (3) without having to connect the pump (3) to the drive (2). When the pump (3) is hooked up to the drive (2), the holding elements (20, 20′) come into abutment with the V-shaped openings (21, 21′) of the pistons (23, 23′) and progressively move the latter until the holding elements (20, 20′) engage in the holes (29, 29′) of the pistons (23, 23′) in the end position. This mechanism makes it possible to hook the pump (3) up to the drive (2) regardless of the position of the holding elements (20, 20′).

According to FIG. 17, the cover (7) comprises two flaps (77, 77′), preferably perpendicular to the cover (7), so as to close the pumping chambers (24, 24′) at their bottom end and thus prevent any foreign elements, e.g., dust, fluid particles, particles from utensils, from getting into the pumping chambers (24, 24′).

### VARIATIONS

A control system for the pump, which is not illustrated, can be added in order to control the pump drive in either a programmable or manual manner. Position sensors may be added to the moving or fixed portions of the system in order to measure displacement, speed, pressure, flow rate, or the force exerted on one or more elements of the system, as well as to ensure proper functioning. Alerts may be transmitted via either wired or wireless connections to an external control system in accordance with the data measured by one of the sensors in order to shut down the system or to inform the operator regarding the usage parameters. The external control system can be adapted so as to control the closed-loop actuator in accordance with the parameters measured by the sensor(s) and an associated algorithm.

The pump, the drive, and the control system can be installed in a box, with a battery or rechargeable battery element optionally included, to form a compact, portable apparatus. One or more control elements such as a pushbutton, a switch, a voice control, gesture or face recognition can be added to the box.

The pump or the box can be adapted for receiving one or more fluid dispensing elements such as a tube, a nozzle, a needle, a mixer, a subcutaneous injection system, an aerosol dispenser, or any other dispensing element.

The pump or the box can be hooked up to a removable vessel containing the fluid to be dosed or to a circuit for continuously supplying optionally pressurized fluid.

The drive can be made in the form of a mechanical assembly capable of being actuated manually in order to obtain an optionally disposable, low-cost fluid propelling device, for example for the dosing of medication contained in an integrated vessel such as a pre-filled cartridge. The apparatus can be disposed of once the cartridge is empty.

The pump and the drive can be integrated in numerous variants of fixed or mobile apparatuses in different forms and employing one or more external elements such as a camera, a laser sight, an acoustic sensor, a gas sensor, a pressure sensor, a temperature sensor, a humidity sensor, a RFID tag, a barcode reader, a proximity sensor, or any other kind of indicator/sensor for targeting or determining a parameter linked to the pumping of fluid.

The system can be adapted so as to have several vessels or fluid supplies at the inlet for dosing different fluids or for rinsing the pump, for example.

The constituent elements of the pump are preferably plastic, metal, or ceramic; they can be obtained by machining, injection, or sintering processes, or any other industrial process.

The sealing elements are preferably thermoformed or overmolded elastomer elements. The gaskets can be O-rings or any other sealing element. The seal between the fixed and mobile portions can also be obtained by aligning or burnishing the parts.

The constituent elements of the pump are preferably plastic and disposable. The pump can be sterilized for dispensing food or medications, for example. However, the choice of materials is not limited to plastics.

The guide elements are preferably rollers or bearings. However, in certain cases they may be omitted or replaced with low friction coefficient and/or lubricated parts.

In another variant of the invention, it is possible to adapt the pump and the drive in such a way that elastic, preferably silicone tubes can be hooked up to the inlet and outlet ports for measuring the pressure at the inlet and outlet of the pump by means of a pressure sensor in contact with the elastic tubes.

In another variant of the invention, it is possible to adapt the pump and the drive so as to have only one piston and one pumping chamber.

In another variant, the pressure on the flexible elements or elastic tubes can be measured with strain gages in direct or indirect contact with the flexible elements or elastic tubes.

Although the invention is described according to several embodiments, there are other variants that are not presented here. The scope of the invention is therefore not limited to the embodiments described above.

1. A pump comprising at least one piston disposed in a pump body, forming at least one pumping chamber having at least one inlet port, at least one outlet port and an axially moving port switch.

2. The pump according to claim 1, in which the pump body comprises two opposed and parallel pumping chambers.

3. The pump according to claim 1, in which the port switch comprises a guide element.

4. The pump according to claim 1, in which the piston comprises a bracket with a V-shaped opening.

5. The pump according to claim 1, comprising a push-piece on its top portion.

6. The pump according to claim 5, comprising at least one reset element positioned between the push-piece and the pump body.

7. The pump according to claim 5, in which the push-piece is attached to the port switch.

8. The pump according to claim 1, in which the inlet and outlet ports are each in contact with a cavity located on the bottom side of the pump body.

9. The pump according to claim 7, in which the cavities are closed by flexible elements.

10. The pump according to claim 1, comprising a cover.

11. The pump according to claim 9, in which the cover is attached to the pump body.

12. The pump according to claim 9, in which the cover comprises at least one opening.

13. The pump according to claim 1, in which the sealing between the mobile portions is achieved with at least one elastomer.

14. The pump according to claim 1, in which the parts are plastic and disposable.

15. The pump according to claim 1, in which the parts are sterilized.

16. The pump according to claim 1, in which the drive comprises at least one pressure sensor.
